(19) United States
(12) Patent Application Publication
(10) Pub. No.: US 2016/0362349 A1
(43) Pub. Date: Dec. 15, 2016

(54) STABLE CONVERSION OF ALKYL HALIDE TO OLEFINS

(71) Applicants: Ashim Kumar GHOSH, Sugar Land, TX (US); Neeta KULKARNI, Sugar Land, TX (US); Mike MIER, Sugar Land, TX (US); SABIC GLOBAL TECHNOLOGIES B.V., Bergen op Zoom (NL)

(72) Inventors: Ashim Kumar Ghosh, Sugar Land, TX (US); Neeta Kulkarni, Sugar Land, TX (US); Mike Mier, Sugar Land, TX (US)

(21) Appl. No.: 15/120,780

(22) PCT Filed: Jun. 30, 2015
(86) PCT No.: PCT/US2015/038542

§ 371 Date: Aug. 23, 2016

Related U.S. Application Data
(60) Provisional application No. 62/020,744, filed on Jul. 3, 2014.

(51) Int. Cl. C07C 1/30; B01J 35/10; B01J 37/28; B01J 29/85

(52) U.S. Cl. CPC C07C 1/30; B01J 29/85; B01J 35/1019; B01J 37/28; C07C 2529/85; B01J 2229/186

(57) ABSTRACT

Disclosed are stable catalysts, and methods for their use, that are capable of producing an olefin from an alkyl halide. The catalysts include a phosphorus-treated silicoalummophosphate (SAPO) having a structure of X/SAPO or X/Z-SAPO, where X includes a non-framework phosphorus and Z is one or more elements from Groups 2A, 3A, IVB, VIB, VIIB, VIII, IB of the Periodic Table, or compounds thereof comprised in the SAPO framework.

### CROSS REFERENCE TO RELATED APPLICATIONS

This application claims benefit to U.S. Provisional Patent Application No. 62/020,744 titled “STABLE CATALYST FOR CONVERSION OF ALKYL HALIDE TO OLEFINS”, filed Jul. 3, 2014. The entire contents of the referenced application are incorporated herein by reference.

### BACKGROUND OF THE INVENTION

A. Field of the Invention

The invention generally concerns silicoaluminophosphate (SAPO) catalysts that have been treated with phosphorus containing compounds. The catalysts have shown to provide stable catalyst performance for a prolonged period of use when compared with the non-treated SAPO catalyst.

B. Description of Related Art

Descriptions of units, abbreviation, terminology, etc. used throughout the present invention are summarized in Table 1.

Light olefins such as ethylene and propylene are used by the petrochemical industry to produce a variety of key chemicals that are then used to make numerous downstream products. By way of example, both of these olefins are used to make a multitude of plastic products that are incorporated into many articles and goods of manufacture. FIGS. 1A and 1B provide examples of products generated from ethylene (FIG. 1A) and propylene (FIG. 1B).

Methane activation to higher hydrocarbons, especially to light olefins, has been the subject of great interest over many decades. Recently, the conversion of methane to light olefins via a two-step process that includes conversion of methane to methyl halide, particularly to methyl mono-halide, for example, to methyl chloride followed by conversion of the halide to light olefins has attracted great attention. Zeolite (e.g., ZSM-5) or zeolite type catalysts (e.g., SAPO-34) have been tried for methyl chloride (or other methyl halide) conversion. However, the selectivity to a desired olefin (e.g., propylene) and the rapid catalyst deactivation for the halide reaction remain the major challenges for commercial success.

One of the most commonly used catalysts in the petrochemical industry is ZSM-5 zeolite. It is a medium pore zeolite with pore size about 5.5 Å and is shown to convert methyl halide, particularly methyl chloride or methyl bromide, to C₂-C₄ olefins and aromatics under methyl halide reaction conditions. Whereas, molecular sieve SAPO-34, an isostructure of chabazite zeolite, having small pore opening (3.8 Å) is shown to convert methyl halide to ethylene and propylene and small amounts of C₄ olefins. However, both catalysts are shown to deactivate rapidly during methyl halide conversion due to carbon deposition on the catalysts.

While the SAPO-34 catalyst has good selectivity for both ethylene and propylene, a major problem with the SAPO-34 catalyst is its lack of stable catalytic performance over prolonged periods of use for the alkyl halide conversion. Notably, the currently available SAPO-34 catalyst show methyl chloride conversion rates of less than 20% after being used for 20 h. Such deactivation of the catalyst requires frequent or continuous catalyst regeneration, or frequent catalyst change-out resulting in inefficient plant operation, or use of more catalysts to produce the desired amounts of ethylene and propylene, which in turn increases the manufacturing costs. Still further, the catalytic material has to be re-supplied in shorter time intervals, which oftentimes requires the reaction process to be shut down. This also adds to the inefficiencies of the currently available SAPO-34 catalysts.

TABLE 1

| Abbreviation | Description |
|---|---|
| Å | Angstrom |
| BET SA | BET (Brunauer-Emmett-Teller) surface area |
| ° C. | degree Celsius |
| ° C./min | degree Celsius per minute |
| cm³/min | cubic centimeter per min |
| g | gram |
| g/cm³ | gram per cubic centimeter |
| h | hour |
| m²/g | meter square per gram |
| mole % | mole percent |
| mmole/g-cat | millimole per gram of catalyst |
| NH₃-TPD | ammonia temperature programmed desorption |
| OD | outer diameter |
| % | percent |
| psig | pound per square inch gauge |
| PVₜₒₜₐₗ | total pore volume |
| XRF | X-ray fluorescence |
| WHSV | weight hourly space velocity |
| wt % | weight percent |

### SUMMARY OF THE INVENTION

A discovery has been made that solves the rapid catalyst deactivation problems associated with many SAPO catalysts without compromising their selectivity for ethylene and propylene production. The preferred SAPO is SAPO-34 having an isostructure of chabazite zeolite and pore opening of 3.8 A. The discovery is premised on treating a SAPO-34 catalyst with one or more phosphorus containing compounds. Such treatment may cause structure modification of the SAPO-34 framework by removal of Si and/or Al from the SAPO-34 framework structure. The phosphorus or phosphorus compounds remain as extra-framework materials (for example, as phosphates) in the micropore structure of SAPO-34 rather than in the framework structure. The removal of Si and Al from the framework of SAPO-34 and the presence of extra framework phosphates change acidic properties of the catalyst resulting in the change in catalytic behavior.

In particular, the present discovery illustrates that the stability of phosphorus treated SAPO-34 or phosphorus treated framework substituted SAPO-34 (e.g., Ti-SAPO-34) is more catalytically stable as demonstrated by slower catalyst deactivation when compared to a non-treated form of the catalyst under the same conditions. It was surprisingly discovered that the catalyst deactivation improved by using a wet-impregnation phosphorus treatment process, preferably with phosphoric acid as the phosphorus source, as compared to a slurry evaporation phosphorus treatment process. Without wishing to be bound by theory, it is believed that the increased stability is achieved by reducing the acidity of the SAPO-34 catalysts to optimal levels. This optimal acidity may slow catalyst deactivation which results in longer catalyst life.

In one aspect of the present invention there is disclosed a catalyst capable of producing an olefin from a methyl halide particularly methyl mono-halide (e.g., methyl chloride). The catalyst can include a phosphorus-treated silicoaluminophosphate (SAPO), specifically SAPO-34 which has the same framework structure of chabazite zeolite. The catalyst can be represented by the structure X/SAPO, where X includes a non-framework (i.e., extra-framework) phosphorus. In a particular instance, SAPO-34 is a framework substituted SAPO-34 whereby framework structure elements (Si, Al, P) are substituted by other elements. This framework substituted SAPO structure is represented by the formula: X/Z-SAPO and Z is one or more elements from Groups 2A, 3A, IVB, VIB, VIIB, VIII, 1B of the Periodic Table, or compounds thereof comprised in the SAPO framework. Examples of elements that can be use are Be, B, Co, Cr, Cu, Fe, Mg, Mn, Ni, Ti, etc., preferably by Ti. The titanium substituted SAPO-34 is denoted as Ti-SAPO-34. In a particular instance, the substituted molecular sieve that is Ti-SAPO-34 is further treated with phosphorus containing compound. Non-limiting examples of phosphorus containing compounds that can be used in the context of the present invention include H₃PO₄, (NH₄)H₂PO₄, or (NH₄)₂HPO₄, or a combination thereof. A preferred phosphorus containing compound is H₃PO₄. The P-treated Ti substituted SAPO-34 is designated as P/Ti-SAPO-34.

In one particular aspect, the P-treated SAPO catalyst of the invention can have a surface area of 250 to 500 m²/g, or preferably 275 to 425 m²/g, or more preferably 300 to 400 m²/g, as determined by BET method using N₂ adsorption at −196° C. Still further, the catalyst may also have bimodal acidity showing two major broad peaks, one with peak maximum between 150° C. and 200° C., and the other with peak maximum between 250° C. and 400° C., as characterized by ammonia temperature programmed desorption (NH₃-TPD) technique. The lower temperature peak is attributed to weak acid sites while the higher temperature peak is attributed to strong acid sites. The amounts of desorbed NH₃ can be less than about 0.20 mmole/g of catalyst for the peak maximum between 150° C. and 200° C., and about 0.25 to about 0.50 or preferably about 0.30 to about 0.45 mmole/g of catalyst for the peak maximum between 250° C. and 400° C. The catalyst can have a weight percent of elemental phosphorus based on the total weight of the catalyst of 20.0 to 23.0, or more preferably from 20.0 to 22.0. The catalysts of the present invention can also be heat treated or calcined in air or in an inert atmosphere. Non-limiting temperature ranges for such heat treatment (calcination) include 200 to 600° C., or more preferably from 400 to 550° C. The heat treatment can be performed for greater than 0.5 h, preferably greater than 2 h or more preferably greater than 5 h and less than 20 h. The catalyst performance or rapid deactivation of the catalysts is improved when compared with the same catalyst that has not been treated with phosphorus compound. In one instance, for example, the phosphorus treated catalysts of the present invention are capable of converting at least 25% of the methyl halide after 20 hours of use at a temperature of 325 to 375° C. or are capable of converting 25 to 40% of the methyl halide after 20 hours of use at a temperature of 325 to 375° C. In some aspects, the catalysts have a selectivity of ethylene and propylene of at least 80% at a temperature of 325 to 375° C. In particular aspects of the present invention, the catalysts are prepared by a wet impregnation method or a slurry evaporation method. In preferred aspects, the wet-impregnation method is used.

Also disclosed is a method for converting alkyl halides to olefins with the phosphorus treated catalysts of the present invention. The method can include contacting any one of the phosphorus treated silicoaluminophosphate (SAPO) catalysts of the present invention with a feed that includes an alkyl halide under reaction conditions sufficient to produce an olefin hydrocarbon product. In some non-limiting aspects, the conditions sufficient for olefin hydrocarbon production include a reaction temperature of between 300° C. and 500° C., preferably between 350° C. and 450° C., space velocity (WHSV) of between 0.5 h⁻¹ and 8 h⁻¹ and at less than 200 psig preferably at less than 100 psig, even more preferably less than 20 psig. The alkyl halide comprised within the feed can have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. The feed can include about 10, 15, 20, 40, 50 mole % or more of an alkyl halide such as methyl halide. In particular aspects, the feed can include about 10 to 30 or about 20 mole % of the alkyl halide. Non-limiting examples of methyl halides include methyl chloride, methyl bromide, methyl fluoride, or methyl iodide, or any combination thereof. In particular embodiment, the alkyl halide is methyl chloride or methyl bromide. The method can further include collecting or storing the produced olefin hydrocarbon product along with using the produced olefin hydrocarbon product to produce a petrochemical or a polymer. Additionally, the used and deactivated catalyst can be regenerated (e.g., after 5, 10, 15, 20, 25, or 30 hours of use, the catalyst can be regenerated).

The decrease of alkyl halide conversion can be attributed to carbon deposition on the SAPO catalyst. The carbon deposition causes the blockage of active sites resulting in decrease of conversion. The spent catalyst can be regenerated by burning of carbon deposited. Such carbon burning can generally be performed by heating the spent catalyst under oxygen preferably diluted oxygen, often used air, at temperature between 400 to 600° C.

In still another embodiment of the present invention there is disclosed a system for producing olefins. The system can include an inlet for a feed that includes an alkyl halide, a reaction zone that is configured to be in fluid communication with the inlet, wherein the reaction zone includes any one of the phosphorus treated SAPO catalysts described herein, and an outlet configured to be in fluid communication with the reaction zone to remove an olefin hydrocarbon product from the reaction zone. During use, the reaction zone can further include the alkyl halide feed and an olefin hydrocarbon product (for example, ethylene, propylene, and/or butylene). The temperature of the reaction zone may range from about 325° C. to 375° C. The system can include a collection device that is capable of collecting the olefin hydrocarbon product.

In yet another aspect of the present invention there is disclosed a method of stabilizing a silicoaluminophosphate (SAPO) catalyst or producing any one of the phosphorus treated silicoaluminophosphate (SAPO) catalysts of the present invention. The method includes treating a silicoaluminophosphate (SAPO) with a phosphorus containing compound with a wet-impregnation or slurry evaporation process to obtain any one of the phosphorus treated silicoaluminophosphate (SAPO) catalysts of the present invention. In either of the wet-impregnation or slurry evaporation processes, the produced catalyst may be further subjected to a heat treatment process (for example, heating or calcining), which can include a temperature of 400 to 600° C. for greater than 2 h and less than 20 h. The heating or calcining step may be followed with washing or rinsing the calcined or heat treated catalyst with an aqueous medium at a temperature less than 100° C., followed by a drying step e.g., 250° C. to 350° C. for greater than 2 h and less than 20 h. The heating or calcining step can be followed with washing or rinsing the calcined or heat treated catalyst with an aqueous medium at a temperature less than 100° C., followed by a drying step e.g., 250 to 350° C. for greater than 2 h, preferably greater than 5 h and less than 20 h.

In the context of the present invention, embodiments 1 through 56 are described. Embodiment 1 is a catalyst capable of producing an olefin from an alkyl halide, the catalyst that includes a phosphorus-treated silicoaluminophosphate (SAPO) having the following structure: X/SAPO or X/Z-SAPO, where X includes a non-framework phosphorus and Z is one or more elements from Groups 2A, 3A, IVB, VIB, VIIB, VIII, 1B of the Periodic Table, or compounds thereof comprised in the SAPO framework. Embodiment 2 is the catalyst of embodiment 1, having the following structure: X/Z-SAPO. Embodiment 3 is the catalyst of embodiment 2, wherein Z is Be, B, Co, Cr, Cu, Fe, Mg, Mn, Ni, or Ti. Embodiment 4 is the catalyst of any one of embodiments 1 to 3, wherein SAPO is SAPO-34. Embodiment 5 is the catalyst of embodiment 1, having the following structure: X/Ti-SAPO-34, where Ti is included in the SAPO framework. Embodiment 6 is the catalyst of any one of embodiments 1 to 5, wherein the phosphorus treated SAPO has been treated with H₃PO₄, (NH₄)H₂PO₄, or (NH₄)₂HPO₄, or an combination thereof. Embodiment 7 is the catalyst of embodiment 6, wherein the phosphorus treated SAPO has been treated with H₃PO₄. Embodiment 8 is the catalyst of any one of embodiments 1 to 7, having a surface area of 250 to 500 m²/g, or preferably 275 to 425 m²/g, or more preferably 300 to 405 m²/g. Embodiment 9 is the catalyst of any one of embodiments 1 to 8, having an acidity showing broad peaks with peak maxima between 150° C. and 200° C. and between 250° C. and 450° C., as characterized by ammonia temperature programmed desorption (NH₃-TPD) technique, wherein peak amounts of desorbed NH₃ is less than about 0.20 mmole/g-catalyst for the peak maxima between 150° C. and 200° C., and 0.25 to 0.50 mmole/g-catalyst for the peak maxima between 250° C. and 400° C. Embodiment 10 is the catalyst of any one of embodiments 1 to 9, having an elemental phosphorus content of 20.0 to 23.0 wt. %, or preferably 20.0 to 22.0 wt. %. Embodiment 11 is the catalyst of any one of embodiments 1 to 7, having: (i) a surface area of 250 to 500 m²/g; (ii) an acidity showing broad peaks with peak maxima between 150° C. and 200° C. and between 250° C. and 450° C., as characterized by ammonia temperature programmed desorption (NH₃-TPD) technique, wherein peak amounts of desorbed NH₃ is less than about 0.20 mmole/g-catalyst for the peak maxima between 150° C. and 200° C., and 0.25 to 0.50 mmole/g-catalyst for the peak maxima between 250° C. and 450° C.; and (iii) a total elemental phosphorus content of 20.0 to 23.0 wt % or preferably 20.0 to 22.0 wt. %. Embodiment 12 is the catalyst of any one of embodiments 1 to 11, wherein said catalyst has been heat treated or calcined at a temperature of 200 to 600° C. Embodiment 13 is the catalyst of any one of embodiments 1 to 12, wherein the catalyst is capable of converting at least 25% of the alkyl halide after 20 hours of use at a temperature of 325 to 375° C., a WHSV of between 0.7 and 1.1 h⁻¹, and pressure of 1 to 3 psig. Embodiment 14 is the catalyst of embodiment 13, wherein the catalyst is capable of converting 25 to 40% of the alkyl halide after 20 hours of use. Embodiment 15 is the catalyst of any one of embodiments 1 to 14, having a selectivity of ethylene, propylene, and butylene of at least 90% after 20 hours of use. Embodiment 16 is the catalyst of any one of embodiments 1 to 15, having a selectivity of ethylene and propylene of at least 80% after 20 hours of use. Embodiment 17 is the catalyst of any one of embodiments 1 to 16, wherein the catalyst is prepared by a wet impregnation or slurry-evaporation method. Embodiment 18 is the catalyst of any one of embodiments 1 to 16, wherein the catalyst is prepared by a wet impregnation method.

Embodiment 19 is a method for converting an alkyl halide to an olefin. The method includes contacting any one of the phosphorus treated silicoaluminophosphate (SAPO) catalysts of embodiments 1 to 18 with a feed that includes an alkyl halide under reaction conditions sufficient to produce an olefin hydrocarbon product. Embodiment 20 is the method of embodiment 19, wherein the catalyst has the following structure: X/Ti-SAPO-34, where Ti is comprised in the SAPO framework. Embodiment 21 is the method of embodiment 20, wherein the catalyst has been treated with H₃PO₄. Embodiment 22 is the method of any one of embodiments 19 to 21, wherein the catalyst converts at least 25% of the alkyl halide after 20 hours of use at a temperature of 325 to 375° C., a WHSV of between 0.7 and 1.1 h⁻¹, and pressure of 1 to 3 psig. Embodiment 23 is the method of embodiment 21, wherein the catalyst converts 25 to 40% of the alkyl halide after 20 hours of use. Embodiment 24 is the method of any one of embodiments 19 to 23, wherein the alkyl halide has the following structure: CₙH₍₂ₙ₊₂₎₋ₘXₘ, wherein: n is an integer from 1 to 5, preferably 1 to 3, more preferably 1; X is Br, F, I, or Cl; and m is an integer less than (2n+2) and is m is from 1 to 3, preferably 1. Embodiment 25 is the method of embodiment 24, wherein the alkyl halide is an alkyl mono halide. Embodiment 26 is the method of embodiment 25, wherein the alkyl mono halide is methyl chloride, methyl bromide, methyl fluoride, or methyl iodide, or any combination thereof. Embodiment 27 is the method of embodiment 24, wherein the alkyl mono halide is methyl chloride. Embodiment 28 is the method of any one of embodiments 25 to 27, wherein the feed includes at least a second alkyl mono halide in an amount less than 10 mole %, preferably less than 1 mole %, relative to the total halide in the feed. Embodiment 29 is the method of any one of embodiments 25 to 27, wherein the feed includes at least 90 mole %, preferably at least 99 mole % of the alkyl mono halide relative to the total halide in the feed. Embodiment 30 is the method of any one of embodiments 24 to 29, wherein the feed includes about 10 mole % or more of the alkyl halide. Embodiment 31 is the method of embodiment 30, wherein the feed further includes inert gas. Embodiment 32 is the method of embodiment 31, wherein the insert gas is N₂ or He, or both. Embodiment 33 is the method of any one of embodiments 19 to 31, further including collecting or storing the produced olefin hydrocarbon product. Embodiment 34 is the method of any one of embodiments 19 to 33, further including using the produced olefin hydrocarbon product to produce a petrochemical or a polymer. Embodiment 35 is the method of any one of embodiments 19 to 34, further including regenerating the used catalyst after 20, 25, or 30 hours of use. Embodiment 36 is the method of any one of embodiments 19 to 35, wherein the catalyst is prepared by a wet impregnation method. Embodiment 37 is the method of any one of embodiments 19 to 36, wherein the catalyst is prepared by a slurry evaporation method.

Embodiment 38 is a system for producing olefins. The system includes: an inlet for a feed that includes an alkyl halide; a reaction zone that is configured to be in fluid communication with the inlet, wherein the reaction zone includes any one of the phosphorus treated silicoaluminophosphate (SAPO) catalysts of embodiments 1 to 18; and an outlet configured to be in fluid communication with the reaction zone to remove an olefin hydrocarbon product from the reaction zone. Embodiment 39 is the system of embodiment 38, wherein the reaction zone further includes the feed and the olefin hydrocarbon product. Embodiment 40 is the system of embodiment 39, wherein the olefin hydrocarbon product includes ethylene and propylene. Embodiment 41 is the system of any one of embodiments 38 to 40, wherein the temperature of the reaction zone is 325 to 375° C. Embodiment 42 is the system of embodiment 41, wherein the WHSV is between 0.7 and 1.1 h⁻¹ and pressure of 1 to 3 psig. Embodiment 43 is the system of any one of embodiments 38 to 42, further including a collection device that is capable of collecting the olefin hydrocarbon product.

Embodiment 44 is a method of stabilizing a silicoaluminophosphate (SAPO) catalyst or producing any one of the phosphorus treated silicoaluminophosphate (SAPO) catalysts of embodiments 1 to 18. The method includes treating a silicoaluminophosphate (SAPO) with a phosphorus containing compound with a wet-impregnation or slurry evaporation process to obtain any one of the phosphorus treated silicoaluminophosphate (SAPO) catalysts of embodiments 1 to 18. Embodiment 45 is the method of embodiment 44, wherein the SAPO is subjected to the wet-impregnation process. Embodiment 46 is the method of embodiment 45, wherein the wet-impregnation process includes: (a) obtaining an aqueous solution of a phosphorus containing compound; (b) obtaining a dry or lyophilized SAPO; and (c) adding the aqueous solution to the dry or lyophilized SAPO to obtain the phosphorus-treated SAPO. Embodiment 47 is the method of embodiment 46, further including adding water after step (c). Embodiment 48 is the method of embodiment 44, wherein the SAPO is subjected to the slurry evaporation process. Embodiment 49 is the method of embodiment 48, wherein the slurry evaporation process includess: (a) obtaining an aqueous solution of a phosphorus containing compound; (b) obtaining a slurry that includes water and SAPO; (c) combining the aqueous solution and the slurry to obtain a mixture; and (d) drying the mixture to obtain the phosphorus-treated SAPO. Embodiment 50 is the method of embodiment 49, wherein the slurry in step (b) is heated to a temperature of 70 to 100° C. Embodiment 51 is the method of any one of embodiments 44 to 50, further including heat treating the phosphorus-treated SAPO at a temperature of 200 to 600° C. for greater than 2 h, preferably greater than 5 h and less than 20 h. Embodiment 52 is the method of embodiment 51, further including treating the heat treated phosphorus treated SAPO with water followed by drying at a temperature of 250 to 350° C. for greater than 2 h, preferably greater than 5 h and less than 20 h. Embodiment 53 is the method of any one of embodiments 44 to 52, wherein the SAPO has the following structure prior to phosphorus treatment: Ti-SAPO-34, where Ti is comprised in the SAPO framework. Embodiment 54 is the method of embodiment 53, wherein the phosphorus treated SAPO has the following structure: X/Ti-SAPO-34, wherein X includes a non-framework phosphorus. Embodiment 55 is the method of any one of embodiments 44 to 54, wherein said phosphorus containing compound is H₃PO₄, (NH₄)H₂PO₄, or (NH₄)₂HPO₄, or an combination thereof. Embodiment 56 is the method of embodiment 55, wherein the phosphorus containing compound is H₃PO₄.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, basic and novel characteristics of the catalysts of the present invention are their ability to selectivity produce light olefins, and in particular, ethylene and propylene, in high amounts, while also remaining stable/activated after prolonged periods of use (e.g., 20 hours).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1A is a chart of various chemicals and products that can be produced from ethylene.

FIG. 1B is a chart of various chemicals and products that can be produced from propylene.

FIG. 2 is a schematic of an embodiment of a system for producing olefins from alkyl halides.

FIG. 3 is a graphical depiction of amount of NH₃ desorbed (mmole/g-cat) versus NH₃ desportion temperature in ° C. for Ti-SAPO-34 and embodiments of phosphorus treated catalysts of the present invention. Curves A, B, C, E and G refer NH₃-desorption curves for Catalysts A, B, C, E and G, respectively.

FIG. 4 is a graphical depiction of CH₃Cl conversion in mole % versus time on stream in hours for Ti-SAPO-34 and embodiments of phosphorus treated catalysts of the present invention. Curves A, B, C and E represent conversions for Catalyst A, B, C and E, respectively.

FIG. 5 is a graphical depiction of CH₃Cl conversion in mole % versus time on stream in hours for embodiments of phosphorus treated catalysts of the present invention. Curves C, G and K represent conversions for Catalyst C, G and K, respectively.

FIG. 6 is a graphical depiction of selectivity of ethylene, propylene and butylene in mole % versus time on stream in hours for embodiments of Ti-SAPO-34 and embodiments of phosphorus treated catalysts of the present invention. Curves 1, 2, and 3 refer to ethylene, propylene and butylene selectivity, respectively, over Catalyst A; and curves 4, 5 and 6 refer to ethylene, propylene and butylene, respectively, over Catalyst C.

### DETAILED DESCRIPTION OF THE INVENTION

The currently available SAPO catalysts, particularly SAPO-34 catalysts, show high activity for alkyl halide conversion with selectivity to light olefins (e.g., ethylene and propylene). These types of catalysts, however, tend to rapidly deactivate when used for a prolonged time-period. This rapid deactivation leads to a number of processing and cost inefficiencies.

A discovery has been made that result in SAPO catalysts having improved stability showing slower catalyst deactivation for converting alkyl halides to light olefins. In particular, it was discovered that treating SAPO catalysts with phosphorus containing compounds via a wet-impregnation method surprisingly improved the catalytic performance stability of the catalysts. This improved stability results in a more efficient and continuous production of light olefins from alkyl halides without having to continuous catalyst regeneration or constantly provide additional catalyst to the reaction process as compared to current non-phosphorus treated SAPO catalysts.

These and other non-limiting aspects of the present invention are discussed in further detail in the following sections.

### A. SAPO Materials

Silicoaluminophosphates (SAPO) materials have an open microporous structure with regularly sized channels, pores or “cages.” Because of their small pore size, these materials are sometimes referred to as “molecular sieves” in that they have the ability to sort molecules (or ions) based primarily on the size of the molecules or ions. SAPO materials are both microporous and crystalline and have a three-dimensional crystal framework of PO₄⁻, AlO₄⁻ and SiO₄ tetrahedra. The empirical chemical composition on an anhydrous basis is:

mR(SiₓAl_{y}P_{z})O₂

where, R represents at least one organic templating agent present in the intracrystalline pore system; m represents the moles of R present per mole of (SiₓAl_{y}P_{z})O₂ and has a value from zero to 0.3; and x, y, and z represent the mole fractions of silicon, aluminum, and phosphorus, respectively, present as tetrahedral oxides.

Non-limiting examples of SAPO materials that can be used in the context of the present invention include SAPO materials that contain eight member ring openings and a pore size from about 3.5 Å to about 4.5 Å (e.g., SAPO-17, SAPO-18, SAPO-34, SAPO-35, SAPO-42, and SAPO-44). The relationship between the SAPO numbers and their structures is mentioned, for example, in Encyclopedia of Inorganic Chemistry, Vol. 8, 4369 (1994). For instance, the IUPAC codes corresponding to SAPO-17, 18, 34, 35, 42, and 44 are ERI, AEI, CHA, LEV, LTA, and CHA, respectively. A preferred SAPO material is SAPO-34. SAPO-34 has the same framework structure of chabazite zeolite. SAPO-34 and processes of making SAPO-34 are disclosed in U.S. Pat. No. 4,440,871, which is incorporated by reference.

In some embodiments, the SAPO framework contains one or more elements from Groups 2A, 3A, IVB, VIB, VIIB, VIII, 1B of the Periodic Table by partly substituting aluminum and/or phosphorus in the framework structure with the appropriate element to obtain substituted-SAPO structures. Framework modified SAPO structures may be represented by the formula: Z-SAPO, where Z is the element that is substituted in the frame work and the hyphen designates that the element is in the framework. Examples of elements from Group 2A include beryllium (Be) and compounds thereof. Examples of elements from Group 3A include boron and compounds thereof. Examples of elements from Group IB include copper and compounds thereof. Examples of elements from Group IVB include titanium, zirconium, hafnium, and compounds thereof. Examples of elements from Group VIB include chromium, molybdenum, tungsten and compounds thereof. Examples of elements from Group VIIB include manganese and compounds thereon. Examples of elements from Group VIII include cobalt, nickel, iron, and compounds thereof. In preferred aspects, SAPO-34 and Ti-SAPO-34 are modified with phosphorus compounds to make the phosphorus modified SAPO-34 or phosphorus modified Ti-SAPO-34 where the added phosphorus can be as extra-framework inside pores of SAPO-34 or Ti-SAPO-34. Processes for making SAPO-34 and Ti-SAPO-34 are disclosed in US Patent Application Publication No. 2012/0159804, which is incorporated by reference. Still further, SAPO-34 and a Ti-modified SAPO-34 can be obtained from a variety of commercial sources (e.g., Clariant International Ltd. (Munich, Germany); ACS Material, Medford, Mass.).

### B. Phosphorus Materials and Treatment Processes

The SAPO material can be modified by treating with phosphorus (P)-containing compounds. Such modified catalysts may be treated to provide a phosphorus content having a weight percent of elemental phosphorus based on the total weight of the catalyst of 19.0 to 23.0 wt. %, or more preferably from 19.7 to 21.8 wt. %, or even more preferably from 20.0 to 22.0 wt. %. Such phosphorus-containing compounds may include, for example, phosphonic, phosphinous, phosphorus and phosphoric acids, salts and esters of such acids and phosphorus halides. In particular, phosphoric acid (H₃PO₄), ammonium di-hydrogen phosphate (NH₄H₂PO₄) and di-ammonium hydrogen phosphate ((NH₄)₂HPO₄) are used as the phosphorus-containing compound to provide a catalyst for the conversion of alkyl halides to light olefins. A preferred phosphorus containing compound is H₃PO₄. The treatment of the SAPO materials with phosphorus reduced the surface are of the phosphorus treated catalyst as compared to the surface area of the starting SAPO materials. Not to be bound by theory, it is believed that the loss of surface area may be attributed to the formation of the various P-species formed inside the SAPO materials pore structure. In some embodiments, the surface area of the P-treated catalyst can be varied by washing with P-treated catalyst with water.

The phosphorus treatment may be carried out by various techniques, which include slurry evaporation and wet impregnation methods. In slurry evaporation, the phosphorus may be incorporated into the catalyst by combining an aqueous slurry of the SAPO material and an aqueous solution of the phosphorus compound to obtain a mixture. The mixture may be heated to facilitate treatment of the SAPO and evaporation of liquids. Heating of the slurry to temperatures of 70° C. and higher is suitable, for example to 100° C. The slurry may also be stirred or agitated during this step to ensure uniform treatment. The zeolite slurry is heated to near complete evaporation of the liquid which can be dried or calcined to form the phosphorus modified SAPO powder or coarse material.

In the wet impregnation method, an aqueous solution of the phosphorus compound is added, such as by spraying, to the dry SAPO powder without forming a slurry. The dry SAPO, which may be initially in the form of a powder, may be mixed with the phosphorus compound. If desired, water may be added to the mixture to facilitate uniform interaction of the P-compound with the SAPO material. The wet-impregnated SAPO material may then be dried or calcined to obtain the phosphorus-modified SAPO powder or particles.

In either of the wet-impregnation or slurry evaporation processes, the produced catalyst may be subjected to a heat treatment process. The heat treatment may include drying and calcining at a temperature of about 200° C. to about 600° C., or more preferable from 400 ° C. to about 550° C. for greater than 0.5 h, greater than 2 h, preferably greater than 5 h and less than 20 h in air or in an inert atmosphere. Heat treatment may be followed with washing or rinsing the calcined or heat treated catalyst with an aqueous medium at a temperature less than 100° C., followed by a drying step (for example, at a temperature from about 250° C. to 350° C.) for greater than 2 h, preferably greater than 5 h and less than 20 h. The resulting catalyst may have a total elemental phosphorus content of about 20.0 wt. % to 23.0 wt. %, or more preferable from about 20.0 wt. % to 22.0 wt. % based on the total weight of the catalyst.

### C. Phosphorus Treated Catalyst

The catalyst resulting from phosphorus treatment is a phosphorus treated SAPO catalyst (X/SAPO, where X is P and is a non-framework component). Another catalyst resulting from the phosphorus treatment is a phosphorus treated Z-SAPO catalyst (X/Z-SAPO where X is P and Z is Be, B, Co, Cr, Cu, Fe, Mg, Mn, Ni, Ti, or compounds thereof comprised in the framework structure of the SiO₄, AlO₄, PO₄ tetrahedra). Specific examples are P/SAPO-34 and P/Ti-SAPO-34.

The resulting phosphorus treated SAPO catalyst and/or phosphorus treated Z-SAPO catalyst may be characterized by surface area and acidity properties. A surface area of the phosphorus SAPO catalyst (for example, a P/Ti-SAPO-34 catalyst) may be about 250 m²/g to about 500 m²/g, or preferably about 275 m²/g to about 425 m²/g, or more preferably about 300 m²/g to about 400 m²/g, as determined by BET method using N₂ adsorption at −196° C. The phosphorus treated SAPO catalyst may exhibit bimodal acidity as characterized by ammonia temperature programmed desorption (NH₃-TPD) technique. During ammonia temperature desorption, the phosphorus treated SAPO catalyst exhibits two major broad peaks, one with peak maximum between 150° C. and 200° C., and the other with peak maximum between 250° C. and 400° C. The lower temperature peak is attributed to weak acid sites while the higher temperature peak is attributed to strong acid sites. The amounts of desorbed NH₃ may be less than about 0.20 mmole/g of catalyst for the peak maximum between 150° C. and 200° C., and about 0.25 to about 0.50, or preferably about 0.30 to about 0.45 mmole/g of catalyst, for the peak maximum between 250° C. and 400° C. The acidity or acid site concentration (mmole/g-cat) of the phosphorus treated SAPO catalyst of the present invention is less than the corresponding acidity for untreated SAPO catalyst. This lower acidity may contribute to the stability of the catalyst and increase the life of the catalyst during use.

### D. Alkyl Halide Feed

The alkyl halide feed includes one or more alkyl halides. The alkyl halide feed may contain alkyl mono halides, alkyl dihalides, alkyl trihalides, preferably alkyl mono halide with less than 10% of other halides relative to the total halides. The alkyl halide feed may also contain nitrogen, helium, steam, and so on as inert compounds. The alkyl halide in the feed may have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. Non-limiting examples of methyl halides include methyl chloride, methyl bromide, methyl fluoride, or methyl iodide, or any combination thereof. In particular aspects, the feed may include about 10, 15, 20, 40, 50 mole % or more of the alkyl halide. In particular embodiments, the feed contains up to 20% of the feed includes an alkyl halide. In preferred aspects, the alkyl halide is methyl chloride. In particular embodiment, the alkyl halide is methyl chloride or methyl bromide.

The production of alkyl halide particularly of methyl chloride (CH₃Cl, See Equation 1 below) is commercially produced by thermal chlorination of methane at 400° C. to 450° C. and at a raised pressure. Catalytic oxychlorination of methane to methyl chloride is also known. In addition, methyl chloride is industrially made by reaction of methanol and HCl at 180° C. to 200° C. using a catalyst. Alternatively, methyl halides are commercially available from a wide range of sources (e.g., Praxair, Danbury, Conn.; Sigma-Aldrich Co. LLC, St. Louis, Mo.; BOC Sciences USA, Shirley, N.Y.). In preferred aspects, methyl chloride and methyl bromide can be used alone or in combination.

### E. Olefin Production

The phosphorus treated SAPO-34 catalysts of the present invention help to catalyze the conversion of alkyl halides to light olefins such as ethylene and propylene. The following non-limiting two-step process is an example of conversion of methane to methyl chloride and conversion of methyl chloride to ethylene and propylene. The second step illustrates the reactions that are believed to occur in the context of the present invention.

$$\begin{matrix}{CH}_{4} + {X}_{2} \to {CH}_{3} X + H X & ( 1 ) \\ 5 {CH}_{3} X {\to }^{P \text{/} Ti - SAPO - 34} {C}_{2} {H}_{4} + {C}_{3} {H}_{6} + 5 H X & ( 2 )\end{matrix}$$

where X is Br, F, I, or Cl. Besides the light olefins the reaction may produce byproducts such as methane, C₄-C₅ olefins and aromatic compounds such as benzene, toluene and xylene.

Conditions sufficient for olefin production (e.g., ethylene and propylene as shown in Equation 2) include temperature, time, alkyl halide concentration, space velocity, and pressure. The temperature range for olefin production may range from about 300° C. to 500° C., preferably ranging 350° C. to 450° C. In more preferred aspects, the temperature range is from 325° C. to 375° C. A weight hour space velocity (WHSV) of higher than 0.5 h⁻¹ can be used, preferably 0.5 preferably between 0.7 and 1.1 h⁻¹. The conversion of alkyl halide is carried out at a pressure less than 200 psig preferably less than 100 psig, more preferably less than 50 psig, even more preferably less than 20 psig. The conditions for olefin production may be varied based on the type or size of reactor.

The reaction can be carried out for prolonged periods of time without changing or re-supplying new catalyst or catalyst regeneration as compared to non-treated SAPO-34 catalysts. This is due to the stability or slower deactivation of the catalysts of the present invention. Therefore, the reaction can be performed for a period until the level of alkyl halide conversion reaches to a preset level (e.g., 30%). In preferred aspects, the reaction is continuously run for 20 h or 20 h to 40 h or longer without having to stop the reaction to resupply new catalyst or catalyst regeneration. The method can further include collecting or storing the produced olefin hydrocarbon product along with using the produced olefin hydrocarbon product to produce a petrochemical or a polymer.

### F. Olefin Selectivity

Catalytic activity as measured by alkyl halide conversion can be expressed as the % moles of the alkyl halide converted with respect to the moles of alkyl halide fed. In some aspects, the catalysts show a combined selectivity of ethylene and propylene of at least 80%, or between 85-90% after 20 hours of use at reaction conditions which includes temperature of 325 to 375° C., WHSV (of alkyl halide) of 0.7 to 1.1 h⁻¹, reactor pressure of less than 20 psig, preferably, less than 5 psig, or more preferably of 1 to 3 psig. The combined selectivity of ethylene, propylene and butylene of P-treated SAPO-34 catalysts of the present invention is at least 90%, or about 95% and 99% after 20 hours of use at a temperature of 325 to 375° C. As an example, methyl chloride (CH₃Cl) is used here to define conversion and selectivity of products by the following formulas:

$$\% {CH}_{3} Cl Conversion = \frac{( {CH}_{3} Cl ) ° - ( {CH}_{3} Cl )}{( {CH}_{3} Cl ) °} \times 100$$

where, (CH₃Cl)° and (CH₃Cl) are moles of methyl chloride in the feed and reaction product, respectively.

Selectivity for ethylene may be expressed as:

$$\% Ethylene Selectivity = \frac{2 ( {C}_{2} {H}_{4} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) +\end{matrix}} \times 100$$

where, the numerator is the carbon adjusted mole of ethylene and the denominator is the sum of all the carbon adjusted mole of all hydrocarbons in the product stream.

Selectivity for propylene may be expressed as:

$$\% Propylene Selectivity = \frac{3 ( {C}_{3} {H}_{6} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) +\end{matrix}} \times 100$$

where, the numerator is the carbon adjusted mole of propylene and the denominator is the sum of all the carbon adjusted mole of all hydrocarbons in the product stream.

Selectivity for butylene may be expressed as:

$$\% Butylene Selectivity = \frac{4 ( {C}_{4} {H}_{8} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) + …\end{matrix}} \times 100$$

Where, the numerator is the carbon adjusted mole of butylene and the denominator is the sum of all the carbon adjusted mole of all hydrocarbons in the product stream.

Selectivity for aromatic compounds may be expressed as:

$$\% Aromatics Selectivity = \frac{6 ( {C}_{6} {H}_{6} ) + 7 ( {C}_{7} {H}_{8} ) + 8 ( {C}_{8} {H}_{10} )}{\begin{matrix}( {CH}_{4} ) + 2 ( {C}_{2} {H}_{4} ) + 2 ( {C}_{2} {H}_{6} ) + 3 ( {C}_{3} {H}_{6} ) + \\ 3 ( {C}_{3} {H}_{8} ) + 4 ( {C}_{4} {H}_{8} ) + 4 ( {C}_{4} {H}_{10} ) + …\end{matrix}} \times 100$$

where, the numerator is the carbon adjusted moles of aromatics (benzene, toluene and xylene) and the denominator is the sum of all the carbon adjusted mole of all hydrocarbons in the product stream.

### G. Olefin Production System

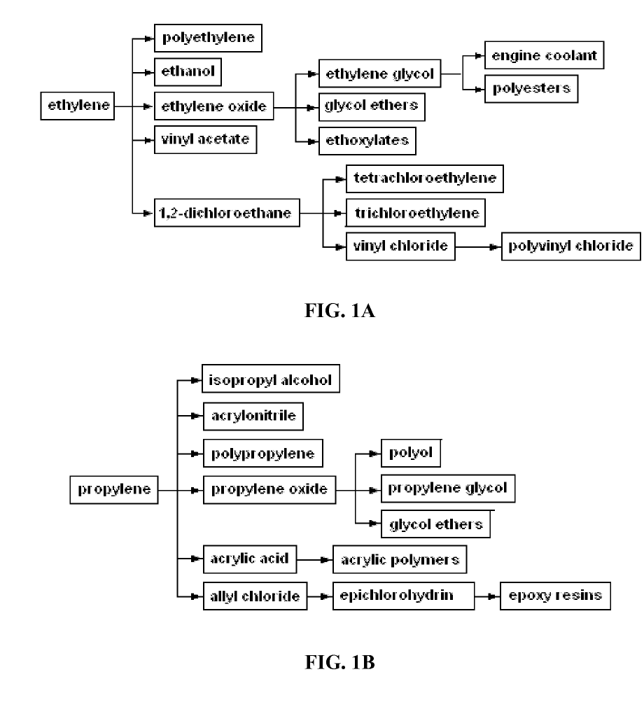

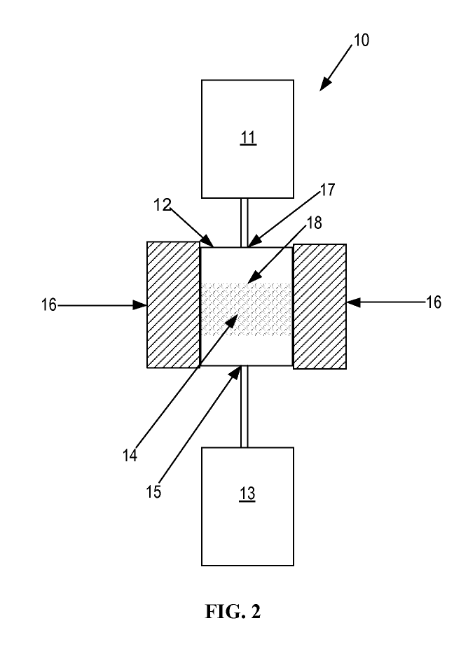

Referring to FIG. 2, a system 10 is illustrated, which can be used to convert alkyl halides to olefin hydrocarbon products with the phosphorus treated SAPO catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the P-treated SAPO catalyst 14 of the present invention. Non-limiting examples of reactors that can be used include fixed-bed reactors, fluidized bed reactors, bubbling bed reactors, slurry reactors, rotating kiln reactors, or any combinations thereof when two or more reactors are used. In preferred aspects, a fixed bed reactor can be used. The amount of the catalyst 14 used can be modified as desired to achieve a given amount of product produced by the system 10. A non-limiting example of a reactor 12 that can be used is a fixed-bed reactor (e.g., a fixed-bed tubular stainless steel reactor which can be operated at atmospheric pressure). The reactor 12 can include an outlet 15 for products produced in the reaction zone 18. The products produced can include ethylene and propylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., ethylene or propylene) for other uses. By way of example only, FIG. 1 provides non-limiting uses of ethylene (FIG. 1A) and propylene (FIG. 1B) produced from the catalysts and processes of the present invention. Still further, the system 10 can also include a heating source 16. The heating source 16 can be configured to heat the reaction zone 18 to a temperature sufficient (e.g., 325° C. to 375° C.) to convert the alkyl halides in the alkyl halide feed to olefin hydrocarbon products. A non-limiting example of a heating source 16 can be a temperature controlled furnace. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

The materials used in the following examples are described in Table 2, and were used as-described unless specifically stated otherwise.

TABLE 2

| Material | Source |
|---|---|
| Ti-SAPO-34^{a} (powder form) | Clariant |
| H₃PO₄ (85 wt % in aqueous) | Sigma Aldrich |
| (NH₄)H₂PO₄ (ammonium dihydrogen phosphate) | Sigma Aldrich |
| (NH₄)₂HPO₄ (diammonium hydrogen phosphate) | Sigma Aldrich |
| CH₃Cl (methyl chloride), 20 mole % (balance N₂) | Praxair |
| Water (deionized) | SABIC labs |

^{a}Ti-SAPO-34 obtained from Clariant International Ltd. (Munich, Germany).

### Preparation of Catalysts A-M

A number of P-treated SAPO-34 catalysts were prepared by using a Ti-SAPO-34 powder and different P-compounds by wet-impregnation and slurry-evaporation methods. The P-compounds used were phosphoric acid and ammonium hydrogen phosphates (e.g., ammonium dihydrogen phosphate and diammonium hydrogen phosphate). In wet-impregnation method the acid or the aqueous solution of the salt was added to the Ti-SAPO-34 powder, mixed thoroughly and water was added to moisten the mixture for uniform mixing. Whereas in slurry method, either the acid or the aqueous solution of the salt was added to aqueous slurry of the Ti-SAPO-34 powder at 90-100° C. and then evaporated the slurry mixture to dryness while stirring.

Catalyst A. A SAPO-34 molecular sieve containing Ti in the framework structure (Ti-SAPO-34) was obtained from Clariant International Ltd. (Munich, Germany). The as-received Ti-SAPO-34 was further calcined in air at about 530° C. using the following calcination temperature profile: step 1—ramp at 5° C./min from room temperature to 120° C. (held 3 h), step 2—ramp at 5° C./min to 350° C. (held 3 h), step 3—ramp at 2° C./min to 530° C. (held 10 h).

Catalyst B. Ti-SAPO-34 was treated with phosphoric acid by a wet-impregnation method. About 4.47 g of H₃PO₄ was slowly sprayed to 30.2 g of as-received Ti-SAPO-34 powder while mixing and about 3 g of water was added to the mixture for homogeneous mixing. The P-treated Ti-SAPO-34 mixture was calcined at 530° C. for 10 h using the same temperature profile used for Catalyst A.

Catalyst C. Catalyst B was further modified by washing the powder Catalyst B in water at 100° C. About 19.6 g of Catalyst B was added in 50 ml water at 100° C. while stirring and maintaining the volume and continued for 2 h, and then filtered to separate the powder catalyst. The powder sample was calcined at 300° C. for 10 h (step 1 was the same as in Catalyst A and in last step ramped at 5° C./min to 300° C.).

Catalyst D. Ti-SAPO-34 was treated with H₃PO₄ by a slurry-evaporation technique. About 30.3 g of as-received Ti-SAPO-34 powder was added in 50 ml water making a slurry while heating and stirring. About 4.5 g H₃PO₄ was added to the slurry when its temperature reached to 90° C. The heating and stirring continued until the liquid from the slurry was slowly evaporated. The P-treated Ti-SAPO-34 was calcined at 530° C. for 10 h using the same temperature profile as used for Catalyst A.

Catalyst E. Catalyst D was further modified by washing the powder Catalyst D in water at 100° C. About 17.1 g of Catalyst D was added in 50 ml water at 100° C. while stirring and maintaining the volume and continued for 2 h, and then filtered to separate the powder catalyst. The powder sample was calcined at 300° C. for 10 h (same as in Catalyst C).

Catalyst F. Ti-SAPO-34 was treated with (NH₄)H₂PO₄ by a wet-impregnation technique. About 4.5 g of (NH₄)H₂PO₄ was dissolved in 10 ml water and the solution was slowly sprayed to 30.3 g of as-received Ti-SAPO-34 powder while mixing. The P-treated Ti-SAPO-34 mixture was calcined at 530° C. for 10 h using the same temperature profile used for Catalyst A.

Catalyst G. Catalyst F was further modified by washing the powder Catalyst F in water at 100° C. About 18.1 g of Catalyst F was added in 50 ml water at 100° C. while stirring and maintaining the volume and continued for 2 h, and then filtered to separate the powder catalyst. The powder sample was calcined at 300° C. for 10 h (same as in Catalyst C).

Catalyst H. Ti-SAPO-34 was treated with (NH₄)H₂PO₄ by a slurry-evaporation technique. About 30.0 g of as-received Ti-SAPO-34 powder was added in 50 ml water making a slurry while heating and stirring. About 4.5 g (NH₄)H₂PO₄ was dissolved in 10 ml water and the solution was added to the slurry when its temperature reached to 90° C. The heating and stirring continued until the liquid from the slurry was slowly evaporated. The P-treated Ti-SAPO-34 was calcined at 530° C. for 10 h using the same temperature profile as used for Catalyst A.

Catalyst I. Catalyst H was further modified by washing the powder Catalyst H in water at 100° C. About 18.0 g of Catalyst H was added in 50 ml water at 100° C. while stirring and maintaining the volume and continued for 2 h, and then filtered to separate the powder catalyst. The powder sample was calcined at 300° C. for 10 h (same as in Catalyst C).

Catalyst J. Ti-SAPO-34 was treated with (NH₄)₂HPO₄ by a wet-impregnation technique. About 5.1 g of (NH₄)₂HPO₄ was dissolved in 10 ml water and the solution was slowly sprayed to 30.0 g of as-received Ti-SAPO-34 powder while mixing. The P-treated Ti-SAPO-34 mixture was calcined at 530° C. for 10 h using the same temperature profile used for Catalyst A.

Catalyst K. Catalyst J was further modified by washing the powder Catalyst J in water at 100° C. About 18.5 g of Catalyst J was added in 50 ml water at 100° C. while stirring and maintaining the volume and continued for 2 h, and then filtered to separate the powder catalyst. The powder sample was calcined at 300° C. for 10 h (same as in Catalyst C).

Catalyst L. Ti-SAPO-34 was treated with (NH₄)₂HPO₄ by a slurry-evaporation technique. About 30.1 g of as-received Ti-SAPO-34 powder was added in 50 ml water making a slurry while heating and stirring. About 5.1 g (NH₄)₂HPO₄ was dissolved in 10 ml water and the solution was added to the slurry when its temperature reached to 90° C. The heating and stirring continued until the liquid from the slurry was slowly evaporated. The P-treated Ti-SAPO-34 was calcined at 530° C. for 10 h using the same temperature profile as used for Catalyst A.

Catalyst M. Catalyst L was further modified by washing the powder Catalyst L in water at 100° C. About 15.0 g of Catalyst L was added in 50 ml water at 100° C. while stirring and maintaining the volume and continued for 2 h, and then filtered to separate the powder catalyst. The powder sample was calcined at 300° C. for 10 h (same as in Catalyst C).

In Table 3, P-compounds and P-treatment methods used for catalysts A to M are summarized. All catalysts were analyzed for Si, Al, P and Ti by XRF techniques and the data are shown in Table 3. The BET surface area (BET SA) and total pore (PV) were measured by BET N₂ adsorption at −196° C. The results are shown in Table 4.

TABLE 3

| | | P-treatment | XRF Elemental Analysis (wt %) | | | |
|---|---|---|---|---|---|---|
| Catalyst | P-compound | Method | Si | Al | P | Ti |
| A | none | none | 3.37 | 23.89 | 18.66 | 2.13 |
| B | H₃PO₄ | Wet-impreg | 3.01 | 21.09 | 21.20 | 1.90 |
| C | H₂O wash of Catalyst B | | 3.32 | 21.69 | 20.23 | 1.96 |
| D | H₃PO₄ | Slurry-evac | 3.05 | 21.65 | 20.66 | 1.96 |
| E | H₂O wash of Catalyst D | | 3.26 | 22.54 | 21.10 | 2.03 |
| F | (NH₄)H₂PO₄ | Wet-impreg | 3.02 | 21.29 | 20.21 | 1.96 |
| G | H₂O wash of Catalyst F | | 3.32 | 21.73 | 21.28 | 1.94 |
| H | (NH₄)H₂PO₄ | Slurry-evac | 3.05 | 21.33 | 20.38 | 1.94 |
| I | H₂O wash of Catalyst H | | 3.19 | 21.74 | 20.97 | 1.95 |
| J | (NH₄)₂HPO₄ | Wet-impreg | 3.01 | 20.92 | 21.46 | 1.88 |
| K | H₂O wash of Catalyst J | | 3.03 | 21.42 | 21.46 | 1.92 |
| L | (NH₄)₂HPO₄ | Slurry-evac | 3.03 | 20.80 | 21.14 | 1.89 |

| | | | | | |
|---|---|---|---|---|---|
| M | H₂O wash of Catalyst L | 3.01 | 21.26 | 20.10 | 1.95 |

^{a}Ti-SAPO-34 obtained from Clariant International Ltd. (Munich, Germany).

The BET surface area of the parent Ti-SAPO-34 was 522 m²/g. In general BET surface area was found to decrease significantly after P-treatment of the SAPO catalyst. The decrease of surface area is dependent on the amount and type of P-compound used and the treatment conditions (e.g., wet-impregnation vs. slurry evaporation method). The loss of surface area may be attributed to the formation of various P-species formed inside the SAPO-34 pore structure. The P-species may be considered as extra-framework material or “debris”. Generally, water treatment (or washing) of the P/Ti-SAPO-34 (particularly, when used wet-impregnation) was found to increase surface area (e.g., Catalyst B vs. Catalyst C).

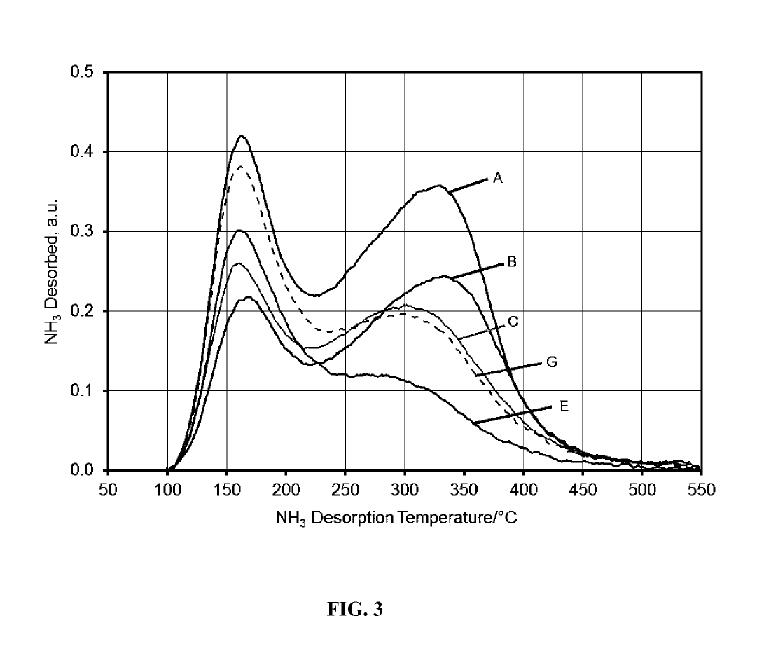

The acidity of the parent Ti-SAPO-34 and P-treated Ti-SAPO-34 were measured by NH₃-TPD. Generally, temperature at which NH₃ desorbed is an estimation of strength of acid sites, e.g., higher the desorption temperature stronger the acid sites. Acidity or acid site density (mmole/g-catalyst) was measured from the amount NH₃ desorbed under the peak and the acidity (with peak maxima) is shown in Table 4. FIG. 3 shows NH₃-TPD of Catalyst A (parent), Catalysts B, C, E and G. Catalyst B was made by treating with H₃PO₄ by wet-impregnation and the Catalyst C was obtained by treating (washing) Catalyst B with water. Catalyst E was a H₃PO₄ treated (slurry-evaporation) and water washed SAPO catalyst. Catalyst G was a NH₄H₂PO₄ treated (wet-impregnation) and water washed SAPO catalyst. The parent and P-treated Ti-SAPO-34 catalyst (catalyst A) shows two peaks—one peak with peak maximum around 159-165° C. and the other peak maximum around 299-337° C. Upon treating the Ti-SAPO-34 with P-compound NH3-desorption peak decreased attributing to the loss of acid sites. The peak maxima also shift to lower temperature suggesting that the loss of stronger sites. Acid site density (mmole/g of catalyst basis) was not much affected by water washing after the P-treatment (Catalysts B and C). The loss of acid sites was more pronounced when the slurry method was used (compare Catalysts C & E).

TABLE 4

| | N₂ Adsorption | | | | |
|---|---|---|---|---|---|
| | BET SA | PVₜₒₜₐₗ | Acidity, mmole/g-cat | | |
| Catalyst | (m²/g) | (cc/g) | weak | strong | total |
| A | 516 | 0.235 | 0.25 (160° C.) | 0.53 (320° C.) | 0.78 |
| B | 341 | 0.207 | 0.15 (164° C.) | 0.42 (337° C.) | 0.57 |
| C | 383 | 0.231 | 0.18 (159° C.) | 0.37 (299° C.) | 0.54 |
| D | 182 | 0.132 | 0.22 (163° C.) | 0.23 (283° C.) | 0.44 |
| E | 188 | 0.137 | 0.20 (160° C.) | 0.21 (283° C.) | 0.41 |
| F | 307 | 0.184 | 0.31 (184° C.) | 0.32 (363° C.) | 0.63 |
| G | 305 | 0.182 | 0.28 (162° C.) | 0.29 (295° C.) | 0.57 |
| H | 321 | 0.191 | 0.30 (186° C.) | 0.33 (366° C.) | 0.63 |
| I | 348 | 0.200 | 0.27 (161° C.) | 0.30 (291° C.) | 0.57 |
| J | 250 | 0.157 | 0.30 (185° C.) | 0.16 (367° C.) | 0.46 |
| K | 243 | 0.164 | 0.25 (162° C.) | 0.27 (293° C.) | 0.52 |
| L | 278 | 0.166 | 0.25 (185° C.) | 0.31 (363° C.) | 0.56 |
| M | 325 | 0.196 | 0.29 (162° C.) | 0.33 (293° C.) | 0.62 |

### Examples 1-8

### (Methyl Chloride Conversion to Olefins)

Catalyst A, B, C, E, G, I, K and M were each tested for methyl chloride conversion by using a fixed-bed tubular reactor at about 350° C. for a period of about 20 h or longer. For catalytic test the powder catalyst was pressed and then crushed and sized between 20 and 40 mesh screens. In each test a fresh load of sized (20-40 mesh) catalyst (3.0 g) was loaded in a stainless steel tubular (½-inch OD) reactor. The catalyst was dried at 200° C. under N₂ flow (100 cm³/min) for an hour and then raised to 300° C. at which time N₂ was replaced by methyl chloride feed (90 cm³/min) containing 20 mole % CH₃Cl in N₂ was introduced to the reactor. The weight hourly space velocity (WHSV) of CH₃Cl was about 0.9 h⁻¹ and reactor inlet pressure was about 1 to 3 psig. Reaction conditions are summarized in Table 5. The reaction temperature was ramped to 350° C. after about 2-3 h of initial reaction period. The pre- and post-run feeds were analyzed and the average was taken into calculations for C-balance and catalyst performance.

TABLE 5
Reaction Conditions of Examples 1-8

| | | | Feed | | Bed | Reactor Inlet |
|---|---|---|---|---|---|---|
| | | Catalyst | Rate¹ | WHSV | Temp | Pressure |
| Example | Catalyst | (g) | (cm3/min) | (h⁻¹) | (° C.) | (psig) |
| 1 | A | 3.01 | 90 | 0.81 | 350 | 1.2 |
| 2 | B | 3.01 | 90 | 0.88 | 352 | 2.7 |
| 3 | C | 3.0 | 90 | 0.88 | 348 | 2.6 |
| 4 | E | 2.99 | 90 | 0.81 | 348 | 1.2 |
| 5 | G | 3.01 | 90 | 0.80 | 349 | 1.2 |
| 6 | I | 3.01 | 90 | 0.87 | 351 | 1.3 |
| 7 | K | 3.01 | 90 | 0.82 | 351 | 1.4 |
| 8 | M | 3.00 | 90 | 0.81 | 348 | 1.5 |

¹Total feed rate (feed contains 20 mole % CH₃Cl in N₂)

Activity of a catalyst was measured as the mole % methyl chloride conversion and selectivity of a product was calculated based on the analyzed products as described earlier. Comparison of methyl chloride conversion at given time-on-stream can give a good comparison of catalyst deactivation. For example, Catalysts A and C show about 31% and 20% conversions, respectively, at 20 h time on-stream, suggesting Catalyst C deactivates significantly slower than that of Catalyst A. In this disclosure, conversions over non-treated and P-treated SAPO-34 catalysts at 20 h on steam are listed in Table 6.

TABLE 6

| | | % Con- | | | | |
|---|---|---|---|---|---|---|
| | | version | % Selectivity at 20 h on-stream | | | |
| | | at 20 h | Ethyl- | Propyl- | Butyl- | |
| Example | Catalyst | on-stream | ene | ene | ene | Total |
| 1 | A | 19.8 | 52.9 | 35.4 | 6.9 | 95.2 |
| 2 | B | 30.0^{a} | 49.0^{a} | 38.8^{a} | 7.0^{a} | 94.8 |
| 3 | C | 32.5^{a} | 48.2^{a} | 39.1^{a} | 8.2^{a} | 95.5 |
| 4 | E | 9.0 | 53.7 | 34.6 | 10.2 | 98.5 |
| 5 | G | 16.2 | 48.9 | 36.3 | 10.4 | 95.6 |
| 6 | I | 11.3^{a} | 54.2^{a} | 32.0^{a} | 8.7^{a} | 94.9 |
| 7 | K | 13.3^{a} | 53.7^{a} | 35.7^{a} | 7.4^{a} | 96.8 |
| 8 | M | 10.4 | 55.0 | 34.4 | 7.9 | 97.1 |

^{a}Data averaged from two test runs.

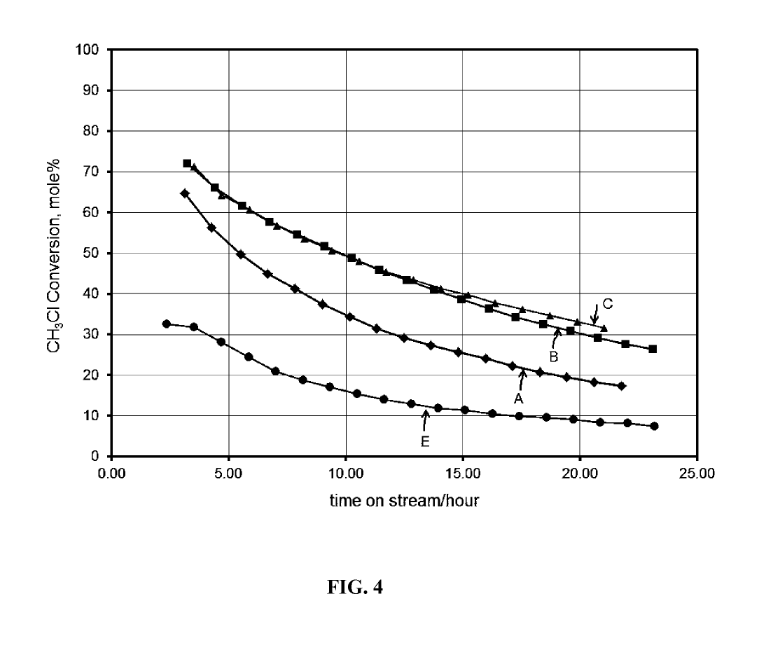

FIG. 4 shows conversion of CH₃Cl over parent Ti-SAPO-34 (Catalyst A) and P-treated Ti-SAPO-34 catalysts (Catalysts B, C, and E). In Table 6 also included are CH₃Cl conversions and product selectivity at 20 h over various catalysts. Catalyst B, a P-treated catalyst (by impregnation method using H₃PO₄), showed higher conversion compared to its parent catalyst, for example CH₃Cl conversion 30.0% for Catalyst B vs. 19.8% conversion for Catalyst A (see Table 6). The increased conversion shown by the P-treated Catalyst B suggests that the P-treatment surprisingly slowed catalyst deactivation. When the Catalyst B was further treated with water by washing it (Catalyst C) showed little improvement in catalyst deactivation showing increased conversion (30.0% conversion for catalyst B vs. 32.5% conversion for Catalyst C). However, Catalyst E (made by slurry evaporation method using H₃PO₄ followed by water-washing) showed unexpectedly poor conversion (9.0% conversion for Catalyst E vs. 19.8% conversion for Catalyst A). The increased conversion for Catalysts B and C may be attributed to optimal acid sites, for example, catalysts containing weak acid sites less than 0.20 mmole/g-catalyst and strong acid sites of about 0.30 to 0.45 mmole/g-catalyst.

Methyl chloride conversions are compared for catalysts made by using the different P-compounds and by using two methods of preparation such as impregnation and slurry-evaporation methods and subsequently P-treated powder catalyst was washed with water. Referring to conversion data in Table 6, the catalyst made by impregnation method (Catalysts C, G and K) show higher conversion than the catalyst made by slurry-evaporation methods (Catalysts E, I, M). Thus, impregnation method is a preferred.

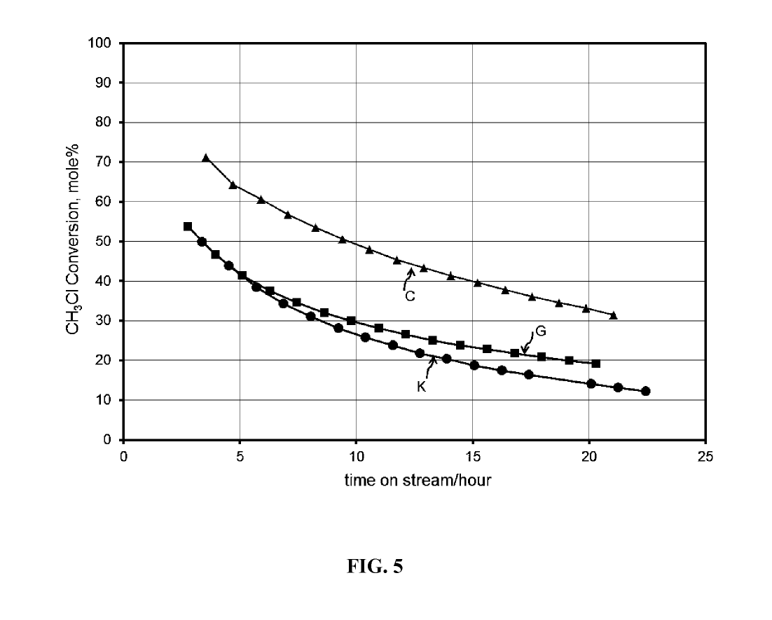

FIG. 5 shows conversion of CH₃Cl over P-treated Ti-SAPO-34 catalysts (Catalysts C, G and K) made by using three P-compounds (H₃PO₄, (NH₄)H₂PO₄, (NH₄)₂HPO₄) and by using impregnation and subsequently P-treated powder catalyst was washed with water. Catalyst C, made by using H₃PO₄, showed higher conversion at a given time-on-stream compared to other two catalysts. Thus, H₃PO₄ is preferred compound.

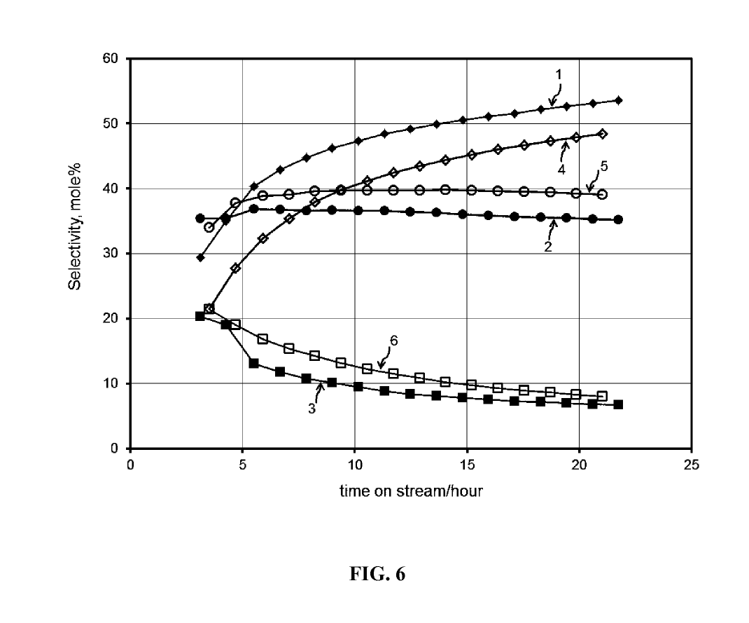

FIG. 6 shows ethylene, propylene and butylene selectivity for non-treated Catalyst A and a P-treated Catalyst C. Table 6 lists ethylene, propylene and butylene selectivity at 20 h (time-on-stream) for all catalysts. In general, ethylene selectivity increases with the decrease of butylene selectivity as the catalyst deactivates (as shown by conversion decreases) with time on stream attributing to coke deposition narrowing the pore opening. At least during the testing period (20-25 h) the propylene selectivity slightly decreased with time. The combined selectivity of ethylene and propylene over the parent and P-treated Ti-SAPO-34 catalysts within the range of 85-89% at 20 h (although conversion varied) with no change in selectivity if compared at constant conversion. The combined selectivity of ethylene, propylene and butylene over the parent and P-treated Ti-SAPO-34 catalysts between about 95% and 99% at 20 h.

From the examples given above P/Ti-SAPO-34 Catalysts B and C which were made by impregnation method using H₃PO₄ as the P-compound show improved catalyst performance or stability after 20 h or longer showing higher methyl chloride conversion without compromising C₂-C₃ olefin selectivity compared to their parent non-treated Catalyst A or other P-treated Ti-SAPO-34 catalysts. The P-treatment by wet-impregnation method and H₃PO₄ as P-compound are preferred for modifying SAPO-34 or substituted particularly Ti substituted SAPO-34 for alkyl halide particularly methyl halide conversion for light olefins.

1. A catalyst capable of producing an olefin from an alkyl halide, the catalyst comprising a phosphorus-treated silicoaluminophosphate (SAPO) having the following structure:
X/Z-SAPO,
where X comprises a non-framework phosphorus and Z is one or more elements from Groups 2A, 3A, IVB, VIB, VIM, VIII, 1B of the Periodic Table, or compounds thereof comprised in the SAPO framework.

2. (canceled)

3. The catalyst of claim 2, wherein Z is Be, B, Co, Cr, Cu, Fe, Mg, Mn, Ni, or Ti.

4. The catalyst of claim 1, wherein SAPO is SAPO-34.

5. The catalyst of claim 1, having the following structure:
X/Ti-SAPO-34,
where Ti is included in the SAPO framework.

6. The catalyst of claim 1, wherein the phosphorus treated SAPO has been treated with H₃PO₄, (NH₄)H₂PO₄, or (NH₄)₂HPO₄, or an combination thereof.

7. The catalyst of claim 6, wherein the phosphorus treated SAPO has been treated with H₃PO₄.

8. The catalyst of claim 1, having a surface area of 250 to 500 m²/g, 275 to 425 m²/g, or 300 to 405 m²/g.

9. The catalyst of claim 1, having an acidity showing broad peaks with peak maxima between 150° C. and 200° C. and between 250° C. and 450° C., as characterized by ammonia temperature programmed desorption (NH₃-TPD) technique, wherein peak amounts of desorbed NH₃ is less than about 0.20 mmole/g-catalyst for the peak maxima between 150° C. and 200° C., and 0.25 to 0.50 mmole/g-catalyst for the peak maxima between 250° C. and 400° C.

10. The catalyst of claim 1, having an elemental phosphorus content of 20.0 to 23.0 wt. %, or preferably 20.0 to 22.0 wt. %.

11. The catalyst of claim 1, having:
(i) a surface area of 250 to 500 m²/g;
(ii) an acidity showing broad peaks with peak maxima between 150° C. and 200° C. and between 250° C. and 450° C., as characterized by ammonia temperature programmed desorption (NH₃-TPD) technique, wherein peak amounts of desorbed NH₃ is less than about 0.20 mmole/g-catalyst for the peak maxima between 150° C. and 200° C., and 0.25 to 0.50 mmole/g-catalyst for the peak maxima between 250° C. and 450° C.; and
(iii) a total elemental phosphorus content of 20.0 to 23.0 wt % or 20.0 to 22.0 wt. %

12-18. (canceled)

19. A method for converting an alkyl halide to an olefin, the method comprising contacting the phosphorus treated silicoaluminophosphate (SAPO) (currently amended) catalyst of claim 1, with a feed comprising an alkyl halide under reaction conditions sufficient to produce an olefin hydrocarbon product.

20. The method of claim 19, wherein the catalyst has the following structure:
X/Ti-SAPO-34,
where Ti is comprised in the SAPO framework.

21. The method of claim 20, wherein the catalyst has been treated with H₃PO₄.

22. The method of claim 19, wherein the catalyst converts at least 25% of the alkyl halide after 20 hours of use at a temperature of 325 to 375° C., a WHSV of between 0.7 and 1.1 h⁻¹, and pressure of 1 to 3 psig.

23. The method of claim 21, wherein the catalyst converts 25 to 40% of the alkyl halide after 20 hours of use.

24-27. (canceled)

28. A method of stabilizing a silicoaluminophosphate (SAPO) catalyst or producing the phosphorus treated silicoaluminophosphate (SAPO) catalyst of claim 1 the method comprising treating a silicoaluminophosphate (SAPO) with a phosphorus containing compound with a wet-impregnation or slurry evaporation process to obtain the phosphorus treated silicoaluminophosphate (SAPO) catalyst of claim 1.

29. The method of claim 28, wherein the SAPO is subjected to the wet-impregnation process, wherein the wet-impregnation process comprises:
(a) obtaining an aqueous solution of a phosphorus containing compound;
(b) obtaining a dry or lyophilized SAPO; and
(c) adding the aqueous solution to the dry or lyophilized SAPO to obtain the phosphorus-treated SAPO.

30. (canceled)

31. The method of claim 30, further comprising adding water after step (c).

32. The method of claim 28, wherein the SAPO is subjected to the slurry evaporation process, and wherein the slurry evaporation process comprises:
(a) obtaining an aqueous solution of a phosphorus containing compound;
(b) obtaining a slurry comprising water and SAPO;
(c) combining the aqueous solution and the slurry to obtain a mixture; and
(d) drying the mixture to obtain the phosphorus-treated SAPO.

33. (canceled)

34. The method of claim 32, wherein the slurry in step (b) is heated to a temperature of 70 to 100° C.
